(19) United States
(12) Patent Application Publication
(10) Pub. No.: US 2003/0187422 A1
(43) Pub. Date: Oct. 2, 2003

(54) Piston

(76) Inventors: Thomas Buch-Rasmussen, Gentofte (DK); Jens Ulrik Poulsen, Virum (DK); Henrik Ljunggreen, Ballerup (DK)

Correspondence Address: Reza Green, Esq., Novo Nordisk Pharmaceuticals, Inc., 100 College Road West, Princeton, NJ, 08540, US

(21) Appl. No.: 10/420,264

(22) Filed: Apr. 22, 2003

Related U.S. Application Data
(63) Continuation of application No. 09/563,572, filed on May 3, 2000, now abandoned.
(60) Provisional application No. 60/133,420, filed on May 11, 1999.

(30) Foreign Application Priority Data
May 5, 1999 (DK) PA 1999 00608

(51) Int. Cl. A61M 5/32

(52) U.S. Cl. 604/413

(57) ABSTRACT

In a medical injection device an ampoule containing a fluid to be injected has a resilient piston closing on one end of the ampoule, which resilient piston is moved forward by a plunger. A membrane closes the other end of the ampoule. An injection needle projecting through the membrane of the ampoule and into the ampoule is mounted prior to use. To protect the pointed end of the needle projecting into the ampoule from being damaged by the resilient piston when the ampoule is about to be empty and the piston is moved close to the membrane, the resilient piston has a central part of the front wall lowered relative to the rest of the front wall to provide a central well in the front wall.

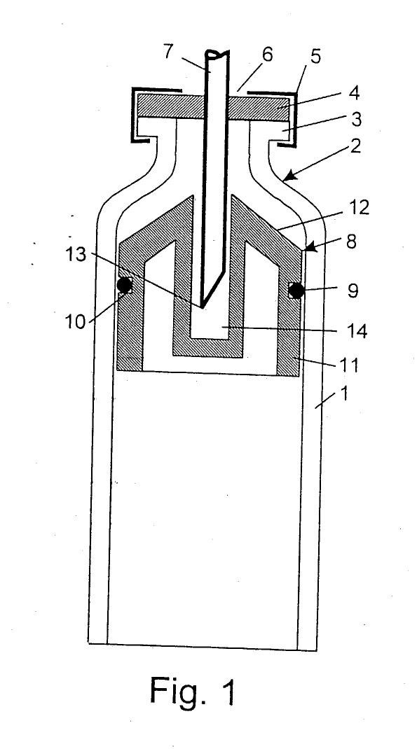

Injection fluids are often stored in vials having the shape of small bottles which are at an outer end of their neck closed by a rubber membrane which is held sealingly against a flange provided at said outer end of the neck. To fill an injection syringe from a vial the syringe is first filled with an amount of air which approximately corresponds to the amount of liquid which is intended to be sucked into the syringe. Thereafter the rubber membrane is pierced by the injection needle of the syringe, the vial is turned upside down to ensure that the liquid in the vial covers the point of the needle. Then the air in the syringe is injected into the vial to provide a super atmospheric pressure in the vial which pressure will help to press the liquid into the syringe when the piston of this syringe is moved outward to fill the syringe.

To avoid the presence of air which influences the storability of the liquid content in the vial negatively and to avoid the necessity for the provision of a super atmospheric pressure in the vial before a syringe can be filled another type of vial has been developed. Such vials still have the shape of a small bottle with a neck part as described above but the bottom of the vial is formed by a piston which can be moved into the vial little by little as the liquid is sucked out of the vial so that the liquid is constantly filling the volume between the piston and the closure at the end of the neck.

Such vials with a piston forming the bottom of the vial can be used for use in a syringe filling device wherein the needle of the syringe is passed through the sealing membrane at the neck of the vial whereafter the piston is pressed into the vial to press some of the content of the vial into the syringe. Alternatively liquid can be sucked out of the vial by drawing the piston of the syringe outward. In both cases the piston of the vial is moved further into the vial. However, as the vial has a relative large diameter the piston of the vial have to be moved only a short distance per unit of the liquid delivered from the vial and the elasticity of commonly used rubber pistons may cause the piston to be deformed in stead of being moved the intended distance. This can be counteracted by the use of a rigid piston fitting with a small play into the vial and being sealed against the inner cylindrical wall of the vial by one or more O-rings in circumferential recesses in the outer cylindrical wall of the piston.

A drawback by vials with a piston is that the piston may damage the pointed end of the needle projecting into the vial when the vial is about to be empty and the piston is close to the neck part. Especially rigid pistons are made of hard materials that can damage the needle and make it unusable for injection of the liquid which is transferred to the syringe which carries the needle.

It is an object to the invention to provide a vial or an ampoule from which liquid can be sucked up through a needle inserted through the sealing membrane without any risk of damaging the needle.

This is obtained by an ampoule comprising a cylindrical vessel which is at a first end closed by a membrane which can in a central area be pierced by a needle and at a second end closed by a piston which can be moved into the ampoule which accommodates a liquid in the variable space between the membrane and a front wall of the piston facing the liquid, which ampoule is according to the invention characterised in that a central part of the front wall of the piston is lowered relative to the rest of said front wall to provide a central well in said front wall of the piston.

By this construction the sharp end of the needle will when it is stuck through the membrane be accommodated in the well in the front wall of the piston if the piston is moved so far into the ampoule that the front wall of this piston lies in a position which can be reached by the needle tip. This way a needle damaging contact with the front wall of the piston is avoided and the needle tip is maintained sharp ready for a piercing of the skin and for injection of the liquid sucked from the ampoule into a syringe carrying the needle.

The piston may be made from a non resilient material and may be sealed against an inner wall of the ampoule by an O-ring positioned in a circumferential recess in a cylindrical wall of the piston.

To minimise the unusable remaining amount in the ampoule the front wall of the piston may be adapted to the shape of the neck part of the ampoule, e.g by having a conical shape.

In the following the invention is described in further details with references to the drawing which shows an ampoule according to the invention.

An ampoule comprising a cylindrical side wall 1 is at a first end terminated by a neck part 2 ending in a circumferential flange 3 against which a piercable membrane 4 is held sealingly by a metal cap 5. At a central part of the membrane 4 the cap 5 has an opening 6 through which the membrane 4 is exposed. An injection needle 7 can be stuck through the membrane 4 to communicate with the inner space of the ampoule in which a liquid is stored between the membrane 4 and a piston 8 which fits into the ampoule and is sealed against the wall 1 by a O-ring 9 positioned in a circumferential recess 10 in a side wall 11 of the piston 8. A front wall 12 of the piston faces the liquid in the ampoule and as this liquid is pressed out through the inserted injection needle 7 the piston 8 is pressed further into the ampoule so that no air pockets appear in the liquid.

When the ampoule is nearly empty and the piston 8 has been pressed so far into the ampoule that the front wall 12 approaches the neck part 2, a risk exists that the sharp tip 13 of the needle 7 may hit against the front wall 12 of the piston 8. To eliminate this risk a well 14 is provided by lowering the central part of the front wall 12 relative to the rest of this front wall. Said well 14 accommodates the end of the needle 7 so that the sharp tip 13 will not hit the piston. The diameter of the well is kept at a minimum so that the amount of liquid which fills the well and cannot be pressed out of the ampoule is minimised. How small said diameter can be depends on how precise the needle is centred by the piercing of the membrane. Especially when the ampoule is made from a plastic material in stead of glass the neck part can be made shorter than usual and a piston with accommodation for the needle tip can be necessitated.

The FIGURE shows an ampoule using a rigid piston with resilient piston rings. Also resilient pistons with integral piston rings can advantageously be provided with a well for accommodation of the needle.

1. An ampoule comprising a cylindrical wall (1) forming a vessel which is at a first end closed by a membrane (4), which can in a central area be pierced by a needle (7), and at a second end closed by a piston (8) which can be moved into the ampoule which accommodates a liquid in the variable space between the membrane (4) and a front wall (12) of the piston (8) facing the liquid, characterised in that a central part of the front wall (12) of the piston (8) is lowered relative to the rest of said front wall to provide a central well (14) in said front wall (12).

2. An ampoule according to claim 1, characterised in that the piston (8) is made from a non resilient material and is sealed against the wall (1) of the ampoule by an O-ring (9) positioned in a circumferential recess (10) in a cylindrical wall (11) of the piston (8).

3. An ampoule according to claim 1, characterised in that the shape of the front wall (12) of the piston (8) is adapted to the shape of the neck part (2) of the ampoule.
